(19) United States
(12) Patent Application Publication
(10) Pub. No.: US 2003/0092967 A1
(43) Pub. Date: May 15, 2003

(54) Laryngoscope or the like

(76) Inventors: Pieter Rousseau Fourie, Durbanville (ZA); Jacobus Adriaan Wessels, Loevensteinville (ZA)

Correspondence Address: JACOBSON HOLMAN PLLC, 400 SEVENTH STREET N.W., SUITE 600, WASHINGTON, DC, 20004, US

(21) Appl. No.: 10/257,361

(22) PCT Filed: Apr. 10, 2001
(86) PCT No.: PCT/IB01/00590

(30) Foreign Application Priority Data
Apr. 14, 2000 (ZA) 2000/1893

(51) Int. Cl. A61B 1/267

(52) U.S. Cl. 600/191

(57) ABSTRACT

A laryngoscope comprising a handle (1) having an axis (5) and carrying a light source (3) of a type having an axis (4) extending in the general direction in which a light beam is emitted thereby is described. The laryngoscope has a removable and replaceable, preferably disposable, blade (7) of translucent material carried by the handle and extending generally transversely relative to the handle axis. The blade has a proximal end (6) and a distal end (13), and the light source is directed transversely relative to the axis of the handle so as to aim the light beam directly at, and generally at right angles to, a transverse light receiving face (11) formed at the proximal end of the blade. A light focusing “lens” (9) may be interposed between the light source and the transverse light receiving face. The handle is preferably made of moulded plastics material and is ergonomically contoured to form a grip which is comfortable to hold; easy for a medical practitioner to position; and which can be effectively gripped.

### FIELD OF THE INVENTION

This invention relates to a laryngoscope or the like and the term laryngoscope is intended, in this specification, to include medical equipment which may not be dedicated to the examination of the larynx or trachea itself or carrying out a procedure associated therewith and which may be employed in examining the oral cavity or throat of a patient. In other words, the invention includes not only a laryngoscope in the strict sense of the word in which case the equipment is usually considered to include what is commonly termed a blade (typically a Macintosh generic blade), but also an illuminated item of equipment used for oral examination and typically employing a spatula for manipulating a patient's tongue.

### BACKGROUND TO THE INVENTION

Medical equipment of the general form of a laryngoscope usually includes a handle housing one or more batteries for energizing a light source associated with the laryngoscope and from which a blade or spatula extends, depending on the exact nature of the equipment being used. In carrying out procedures, or an examination, it is important that adequate light is available in the relevant locations and also that the blade or spatula itself can be easily seen in order to manipulate it correctly without inflicting unnecessary discomfort on the patient.

With this object in view various types of laryngoscopes have been proposed. U.S. Pat. No. 4,947,896 to Robert L Bartlett describes a laryngoscope having a blade with electrical conductors running in service channels down the blade to lamps positioned at spaced locations along its length. This arrangement is complicated; does not lend itself-particularly to the provision of disposable blades; and, does not lend itself particularly to be effective and simple as regards sterilization. U.S. Design Pat. No. 0,413,977 illustrates a different laryngoscope blade which also has a lamp positioned intermediate its ends.

Other prior proposals have used light sources enclosed within a transparent blade, or handle which blends into a blade. U.S. Pat. No. 5,879,304 to Aaron Schuchman et al describes a hollow translucent blade which may be of a disposable variety and which receives an optuionally removable assembly including conductors and a lamp positioned intermediate the ends of the blade. This arrangement is considered, by applicant, to be inappropriate to disposable blades because of the necessity to remove the lamp subassembly prior to disposing of the blade, or, on the other hand, disposing of the lamp subassembly together with the blade which becomes rather costly. The arrangement furthermore does not r necessarily distribute the light adequately to all required positions.

European patent application EP 1,025,796 to Gruhan Technologies Inc describes another variety of laryngoscope in which the free or distal end of the blade is illuminated by a light source within the body of the laryngoscope. The light distribution means in the form of a roughened surface portion or an integral Fresnel lens formed in the surface of the blade is suggested to emit some of the light traveling down the blade at a position short of the free or distal end thereof. This patent describes, as an alternative, a disposable variety of blade with reference to FIG. 10 of its drawings but the illumination of the disposable blade is not discussed at all. The other embodiments of the invention would be inconvenient and costly to render disposable.

British patent 2,296,436 to Peter William King-Lewis et al describes an optionally disposable laryngoscope blade having an integral socket formed at one end, the proximal end, for receiving a part of a handle having a light source extending at generally right angles to the length of the blade. The light is reflected Into the translucent blade by a reflecting surface at an angle of about 45 degrees to the axis of the handle to reflect the light rays through an angle of about 90 degrees. This patent also proposes, without providing any detail, that internal reflecting services could be employed to deliver light at a number of points along the length of the blade. The blade suggested by this patent is, because of the construction of the socket and its means of attachment to the handle, rather large and, accordingly, expensive at least from the point of view of cost of materials and also possibly tools and dies. A similar blade is described in published international patent application WO 01/10293 to the same applicant.

It has also been proposed in a number of patents to use optical fibres for transferring light towards the distal end of the laryngoscope blade and U.S. Pat. No. 5,584,795 to Elio Valenti is one example of such a proposal. Such an arrangement also does not necessarily achieve adequate light distribution and furthermore does not lend itself to the provision of a simple construction providing a simple and costeffective disposable laryngoscope blade.

U.S. Pat. No. 4,320,745 describes a laryngoscope blade in which a light source at an end of one tongue of a light conducting blade receives light that is transmitted alone the length of the blade to become emitted from the distal end thereof. The difficulty here is that inadequate light may be present in positions other than the distal end region and this may impede a medical practitioner in manipulating the laryngoscope.

It will be understood from the aforegoing that applicant considers the provision of a disposable type of laryngoscope blade to be highly desirable but that such a blade should be capable of delivering adequate light to locations where it is required in use.

Also, existing laryngoscopes are usually dedicated to one type of function, that is, to use as a laryngoscope In the strict sense of the word. Other dedicated items of illuminated medical equipment are generally used for the examination of the oral cavity and throat.

### OBJECT OF THE INVENTION

It is an object of this invention to provide a laryngoscope which can be configured to exhibit improved illumination, has a blade construction which lends itself to being made as a disposable item, and which may, if required, provide for additional versatility of a single item of equipment.

### SUMMARY OF THE INVENTION

In accordance with a first aspect of the invention there is provided a laryngoscope comprising a handle having an axis and carrying a light source of a type having an axis extending in the general direction in which light emitted by the light source travels in operation, a removable and replaceable blade of translucent material carried by the handle and extending generally transversely relative to the handle axis, the blade having a proximal and a distal end, and means for directing light emitted by the light source into and down the blade[, the laryngoscope being characterized in that] with the axis of the light source [is] directed transversely relative to the axis of the handle and directly at a transverse light receiving face formed at the proximal end of the blade, the laryngoscope being characterized in that the blade has a plurality of light emitting windows or lenses spaced apart along its length.

Further features of the invention provide for the light receiving face to be at generally right angles to the axis of the light source; for a light focusing “lens” (as herein further described below) to be interposed between the light source and the transverse light receiving face formed at the proximal end of the blade; for the said light receiving face to be approximately circular in shape and of approximately the same diameter as the light source itself or light focusing “lens”; for said “lens” to be a short translucent substantially cylindrical formation generally coaxial with the light source and light receiving face; for the light source to be a small diameter Xenon bulb preferably having a narrow angle of focus or a light emitting diode (LED) of a suitable colour, typically white, and also preferably having a narrow angle of focus, say of the order of 15°; and for there to be optionally two or more light sources in which case at least one of them is directed at the said transverse light receiving face whilst the other or others may either have a light receiving face associated therewith or may be used for more general lighting not being required specifically as additional light to be channeled down the blade.

Further features of this aspect of the invention provide for the blade to have a plurality of light emitting windows or lenses spaced apart along its length; for at least one light emitting window or lens to be formed at or adjacent the distal or terminal end of the blade with at least one other being defined by a transverse surface formed on one side of the blade by way of a step change in thickness of the blade and directed in the general direction of the length of the blade; for the blade to have just two light emitting windows, one between approximately one half and, one quarter of the length of the blade from its distal end and the other at the distal end thereof; and for the blade to be disposable in nature.

Still further features of this aspect of the Invention provide for the blade to be formed integral with an attachment unit assuming the general form of a hood adapted to releasably clip over a portion of an end of the handle, preferably with a wedge type of locking action to align the light receiving face of the blade with the light source; and for the hood to include an inwardly directed flange extending in the general direction of the blade and cooperating with a groove in the handle to guide movement of the hood relative to the handle in a direction transverse to the axis of the handle and to positively locate the hood relative to the handle in the installed position for which purpose the flange is preferably inclined slightly relative to an opposite wall of the hood to form a wedge type of locking action with the handle.

The blade and hood is preferably made as a single piece injection moulding of an optical grade of acrylic plastics material.

It is a particular, although optional, aspect of the invention that the handle have a separate docking formation offset from but near the light source for receiving, as an alternative to said laryngoscope blade, a spatula extending outwards from the handle at generally right angles thereto with the light source located on the side of the spatula remote from the major portion of the handle.

Further features of this aspect of the invention provide for the spatula to be made of translucent material; and for there to be means adapted to direct a portion of the light emitted by the light source into and through the spatula in a direction along the length thereof so as to render same at least visible to some extent, in use.

Still further features of this aspect of the invention provide for the “lens” to be adapted to direct a major portion of the light emitted by the light source in the aforesaid direction with a small amount being directed to the spatula; and for a clipping unit to engage the spatula operatively to retain it in position in which case the clipping unit is transparent and preferably integral with the “lens”.

Preferably the spatula is disposable in nature, as in the case of the blade, so that sterilization procedures are minimized.

In all variations of the invention the handle preferably houses replaceable batteries, or a rechargeable battery, and the handle carries a switch for controlling the electrical power supplied to the light source. Furthermore, the handle is preferably ergonomically contoured to form a grip which is comfortable to hold; easy for a medical practitioner to position; and which can be effectively gripped. The handle is conveniently made of injection moulded plastics material.

Laryngoscope blades adapted to be used in a laryngoscope as defined above are also an important feature of the invention.

In order that the above and other features of the invention will be more fully understood one embodiment thereof which can be selectively used with a blade as a laryngoscope or with a spatula as an item of medical examination equipment will now be described with reference to the accompanying drawings.

### BRIEF DESCRIPTION OF THE DRAWINGS

In the drawings:

FIG. 1 is a perspective illustration of one embodiment of laryngoscope comprising a handle and laryngoscope blade attached thereto;

FIG. 2 is an exploded view of the laryngoscope illustrated in FIG. 1;

FIG. 3 is a sectional elevation of the laryngoscope illustrated in FIG. 1 taken through the light source which is offset from the central plane of the laryngoscope;

FIG. 4 is a sectional elevation of the laryngoscope illustrated in FIG. 1 taken through the central plane of the laryngoscope;

FIG. 5 is an enlarged perspective view of a “lens” and integral clipping unit of the laryngoscope illustrated in FIG. 1;

FIG. 6 is a sectional detail taken through the attachment unit of the laryngoscope blade, being a hood formation, along line VI-VI in FIG. 8;

FIG. 7 is a cross-sectional detail of the laryngoscope blade taken along line VII-VII in FIG. 6;

FIG. 8 is an end view of the laryngoscope blade taken along the line of arrow “A” as shown in FIGS. 2 and 6;

FIG. 9 is a perspective view of the handle alone in an orientation appropriate to use as an item of oral examination equipment;

FIG. 10 is a view similar to FIG. 9 but showing a spatula in operative association with the handle; and,

FIG. 11 is a view similar to FIG. 3 but with a spatula in position as shown in FIG. 10.

### DETAILED DESCRIPTION WITH REFERENCE TO THE DRAWINGS

In the embodiment of the invention illustrated in the drawings the laryngoscope comprises a hollow handle (1) made up basically of two injection moulded parts and which contains replaceable batteries (2) and a light bulb (3) at the one end thereof. The light bulb has its axis (4) directed transversely, and at generally right angles to the axis (5) of the handle so that a light beam emitted by it will be orientated in the general direction in which the adjacent part (6) of blade (7) of the laryngoscope extends. The light bulb (3) is connected to the battery by way of an externally operable switch (8) on the handle.

The light bulb may be any suitable bulb which preferably generates a reasonably narrow beam and typically, a xenon bulb of known type would be appropriate. However, it is also envisaged that light emitting diodes (LED's) of a suitable nature, in particular the so-called white LED's having a suitable light emission angle of say 15 degrees, will be eminently suitable for the purpose. Depending on the exact properties of the light bulb a “lens” (9) is positioned coaxially with the light bulb and immediately in front of it, the “lens” serving to channel the light rays to a somewhat more parallel relationship relative to the light bulb axis. In this embodiment of the invention the “lens” is simply a short cylindrical body of translucent and colourless plastics material formed, in this case, integral with a clipping unit (10) which will be more fully described below.

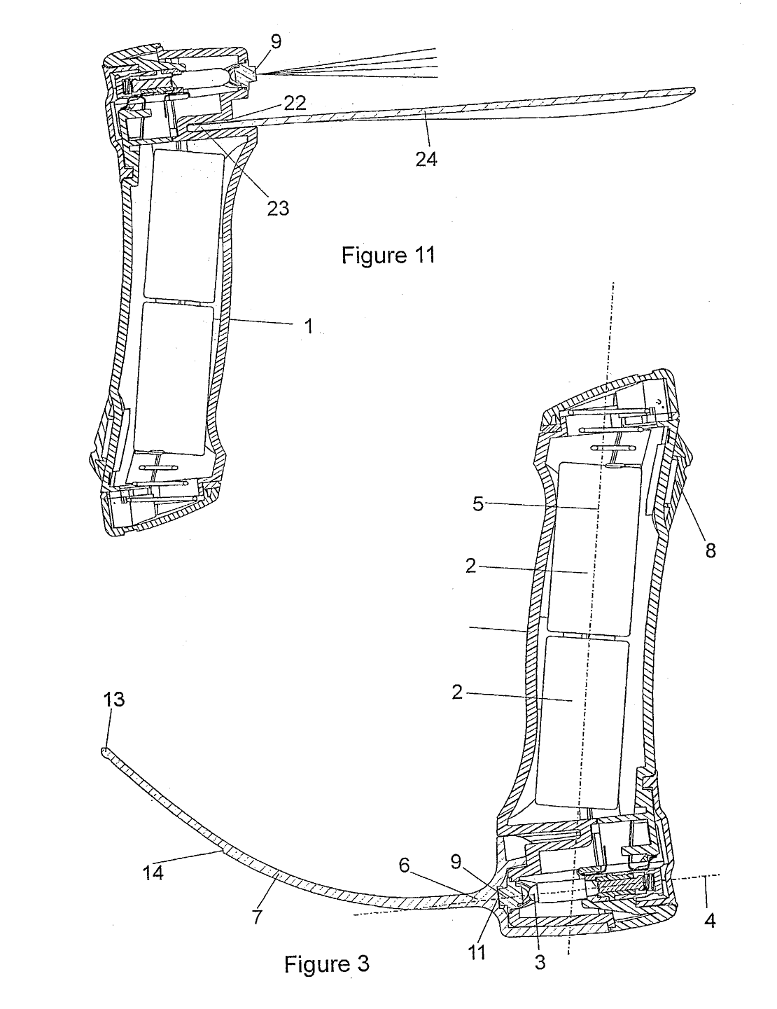

The end of the “lens” remote from the light bulb is directed coaxially at, and is in close proximity to, a similarly shaped light receiving face (11) formed at the inner end of the laryngoscope blade itself and orientated at substantially right angles to the length of the blade at that position (see most clearly in FIGS. 3, 6 and 8). In this manner light from the light bulb is channeled directly into the blade in the direction in which it extends.

The blade is, of course, injection moulded in a totally transparent and colourless material and as a preferred material there is used an optical grade of a suitable acrylic material so that a predominant amount of the light emitted by the light bulb will be conducted down the blade in the required manner. A minor amount of the light becomes diffused into the clipping unit and its destiny will become apparent from the following.

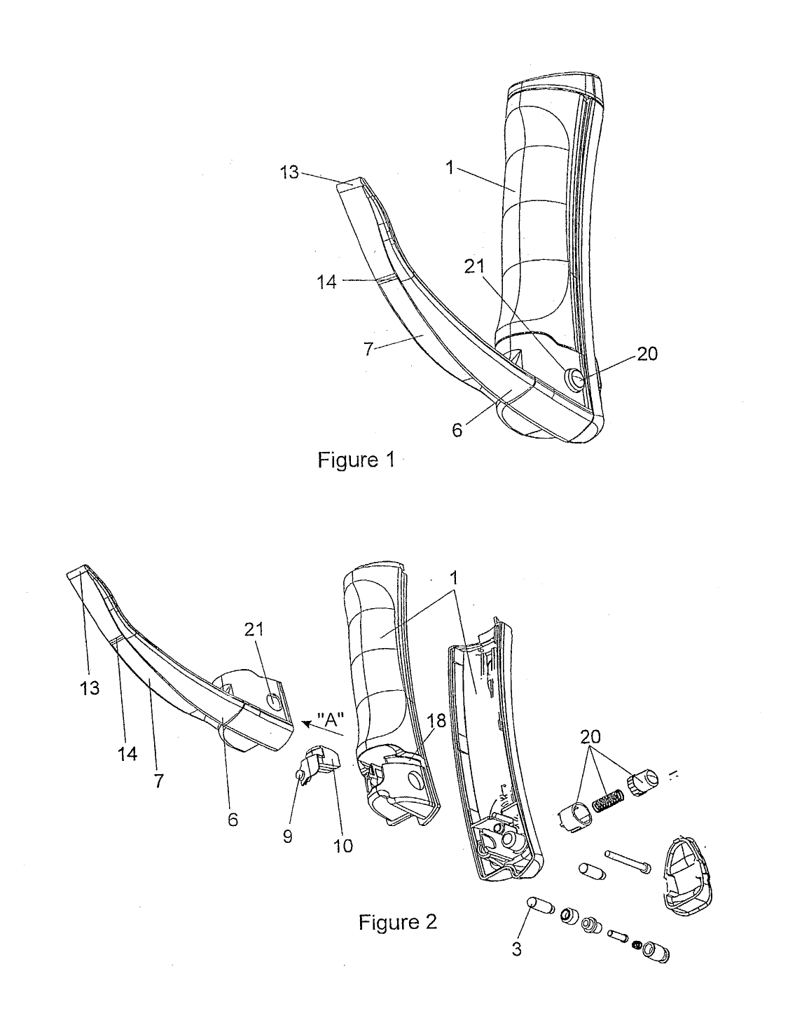

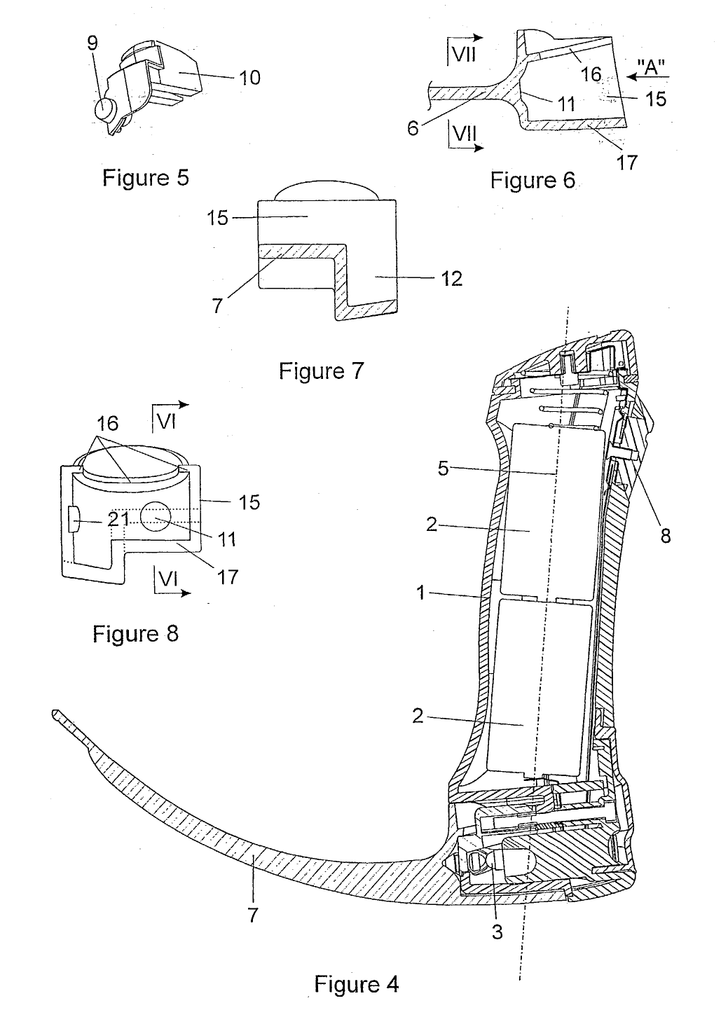

The blade, in this embodiment of the invention, has a substantially conventional general configuration being of arcuate shape in its length, as illustrated in FIGS. 1, 2, and 3. In cross-section, as shown clearly in FIG. 7 the blade has a channel (12) for accommodating, guiding and protecting an endotracheal tube or the like to one side of the blade itself. As a result of this configuration the light source is in a plane offset to one side of the central plane and illustrated in FIG. 3 whilst the central plane is illustrated in FIG. 4.

As provided by this invention, the blade has two light emitting windows (13) and (14) (see in particular FIG. 3). The one light emitting window (13) is formed at the distal end of the blade, in the usual way, whilst the other light emitting window (14) is formed at about one third of the length of the blade from the distal end. The latter light emitting window is formed by creating a step decrease in the thickness of the blade at the position where the window is formed so that the window is located in a plane transverse to the length of the blade.

It has been found in practice that a laryngoscope and blade assembly configured as described above operates particularly effectively with regard to illumination. The transmission of the light substantially directly from the light bulb into the end of the blade in a direction in line with such end of the blade and with the aid, as may be required, of the “lens” minimises losses and enables substantially all of the light to be rendered usable. Any effective amount of light is emitted from each of the two windows which act as lens is and stray light which is last from the general surface of the blade pins to provide sufficient illuminate of the blade to enable it to be easily seen and therefore effectively manipulated by a medical practitioner.

The blade (7) is, in this case, made integral with an attachment unit in the form of a hood (15) configured to clip onto the end of the handle, and in so doing, to cover the front half of the end of the handle. In order to ensure that the blade is effectively rigid, in use, relative to the handle, a wedging action is provided to ensure that the blade is effectively locked relative to the handle in the installed position. To this end the hood (15) has an inwardly directed flange (16) opposite the wall (17) thereof adapted to cover a half of the end of the handle. That wall (17) extends roughly parallel to the part (6) of the blade (7) adjoining it. The flange (16) and opposite wall (17) converge somewhat towards the inner end of the hood where the light receiving face (11) is located.

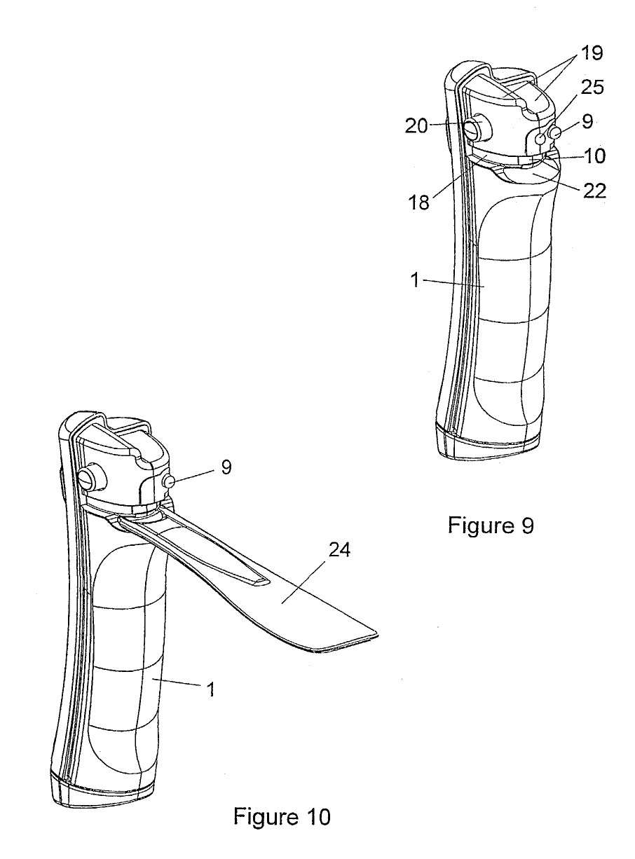

The flange is received in a groove (18) in the side of the handle (shown clearly in FIG. 9) and the opposite wall (17) cooperates with the end face (19) of the handle. The hood is maintained in its installed position by means of a spring loaded catch (20) on one side of the handle and which cooperates with a hole (21) provided in the side of the hood (see FIGS. 1 and 2). The catch can also be slightly tapered to ensure that an effective wedge lock is achieved and that substantially no play exists between the blade and handle in the installed position.

The blade and integral hood is made to be disposable, as indicated above, so as to avoid re-sterilization procedures. It will be understood that blades can be made in various sizes and each different size of blade can be employed with the same handle.

Turning now to the optional second aspect of the invention, the handle in this particular embodiment of the invention has a docking formation in the form of a slot (22) between the light bulb and battery containing portion of the handle as shown in FIGS. 9 to 11. The slot (22) is adapted to receive an end (23) of a translucent and colourless plastic spatula (24). The translucent clipping unit (10) described above actually contacts the end (23) of the spatula, and in so doing, provides a transverse path for a portion of the light emitted by the light bulb and which diffuses into the clipping unit. This small amount of light travels down the spatula to render it visible in use whilst the bulk of the light will be beamed from the “lens” over the top of the spatula as will be apparent from FIG. 11 in particular.

It will be understood that, in use, the spatula can be employed for an oral or throat examination and that the light source will be directed by the “lens” (9) in a manner enabling an examination to take place easily whilst, simultaneously, the light source causes illumination of the spatula to an extent that it can be easily seen and thus manipulated in order to achieve the required examination.

It will also be understood that both the spatula can also be inexpensively injection moulded from plastics material and can, accordingly, be made as disposable items to avoid re-sterilization.

It will also be understood that the handle, one or more laryngoscope blades and one or more spatulas can be packaged as a set with the advantage that only one basic item of equipment is employed thereby not only saving in costs of acquiring equipment but also facilitating transport and storage in the case of mobile medical units or doctors who have to visit patients.

Many variations may be made to the embodiment of the invention described above without departing from the scope hereof. In particular, it is not necessary that the handle be formed to serve in the second aspect of the invention and in this case the slot could be omitted and the lens, if it is present at all, could be made independently of any clipping unit. There may be more than one light source with one, for example, being directed at the light receiving face, and another such as that indicated by numeral (25) in FIG. 9 being arranged to provide more general and diffuse lighting, in use.

Also, the blades themselves formed with at least a light receiving face and optionally two light emitting windows are intended to fall in the scope of the invention.

1. A laryngoscope comprising a handle (1) having an axis (5) and carrying a light source (3) of a type having an axis (4) extending in the general direction in which light emitted by the light source travels in operation, a removable and replacable blade (7) of translucent material carried by the handle and extending generally transversely relative to the handle axis, the blade having a proximal end (6) and a distal end (13), and means for directing light emitted by the light source into and down the blade[, the laryngoscope being characterized in that] with the axis of the light source [is] directed transversely relative to the axis of the handle and directly at a transverse light receiving face (11) formed at the proximal end of the blade, the laryngoscope being characterized in that the blade has a plurality of light emitting windows or lenses (13, 14) spaced apart along its length.

2. A laryngoscope as claimed in claim 1 in which the light receiving face is at generally right angles to the axis of the light source.

3. A laryngoscope as claimed in either one of claims 1 or 2 in which a light focusing “lens” (9) is interposed between the light source and the transverse light receiving face formed at the proximal end of the blade.

4. A laryngoscope as claimed in any one of the preceding claims in which the said light receiving face is approximately circular in shape and of approximately the same diameter as the light source itself or light focusing “lens”.

5. A laryngoscope as claimed in either one of the claims 3 or 4 in which the “lens” is a short translucent, substantially cylindrical formation generally coaxial with the light source and light receiving face.

6. A laryngoscope as claimed in any one of the preceding claims in which the light source is selected from a small diameter Xenon bulb and a light emitting diode (LED).

7. A laryngoscope as claimed in any one of the preceding claims in which there are at least two light sources (3, 25) in which case at least one of them is directed at the said transverse light receiving face whilst the other may be arranged to provide more general lighting not being required specifically as additional light to be channeled down the blade.

8. (Deleted)

9. A laryngoscope as claimed in claim 8 in which one light emitting window or lens (13) is formed at or adjacent the distal end of the blade with at least one other (14) being defined by a transverse surface formed on one side of the blade by way of a step change in thickness of the blade.

10. A laryngoscope as claimed in either one of claims 8, or 9 in which the blade has just two light emitting windows, one being located between approximately one half and one quarter of the length of the blade from its distal end and the other being located at the distal end thereof.

11. A laryngoscope as claimed in any one of the preceding claims in which the blade is formed integral with an attachment unit assuming the general form of a hood (15) adapted to releasably clip over a portion of an end of the handle to align the light receiving face of the blade with the light source.

12. A laryngoscope as claimed in claim 11 in which the hood cooperates with the handle with a wedge type of locking action.

13. A laryngoscope as claimed in claim 11 in which the hood includes an inwardly directed flange (16) extending in the general direction of the blade and cooperating with a groove (18) in the handle to guide movement of the hood relative to the handle in a direction transverse to the axis of the handle so as to positively locate the hood relative to the handle in the installed position, the flange being inclined slightly relative to an opposite cooperating wall (17) of the hood to form the wedge type of locking action with the handle.

14. A laryngoscope as claimed in any one of claims 11 to 13 in which the handle has a spring loaded catch (20) cooperating with the hood to retain it releasably in position on the handle.

15. A laryngoscope as claimed in any one of the preceding claims in which the handle has a separate docking formation (22) offset from but near the light source for receiving, as an alternative to said laryngoscope blade, a spatula (24) extending outwards from the handle at generally right angles thereto with the light source located on the side of the spatula remote from the major portion of the handle.

16. A laryngoscope as claimed in claim 15 in which the spatula is made of translucent material and means (10) are provided for directing a portion of the light emitted by the light source into and through the spatula in a direction along the length thereof so as to render same at least visible to some extent, in use.

17. A laryngoscope as claimed in claim 15 in which said means is a clipping unit adapted to engage the spatula operatively to retain it in position, the clipping unit being transparent and, where a “lens” is present, integral with the “lens”.

18. A laryngoscope as claimed in any one of the preceding claims in which the handle houses replaceable batteries, or a rechargeable battery and the handle carries a switch for controlling the electrical power supplied to the light source.

19. A laryngoscope as claimed in any one of the preceding claims in which the handle is made of moulded plastics material and is ergonomically contoured to form a grip which is comfortable to hold; easy for a medical practitioner to position; and which can be effectively gripped.

20. A laryngoscope blade for use in a laryngoscope as claimed in any one of claims 1 to 14 and having the features defined therein.

21. A spatula for use in a laryngoscope as claimed in any one of claims 15 to 17 and having the features defined therein.

22. A laryngoscope handle for use in a laryngoscope as claimed in any one of claims 1 to 19 and having the features defined therein.
